(19) United States
(12) Patent Application Publication
(10) Pub. No.: US 2026/0257004 A1
(43) Pub. Date: Sep. 3, 2026

(54) NEGATIVE PRESSURE WOUND THERAPY DRESSINGS INCLUDING IN-VIVO OPTICAL SENSING

(71) Applicant: Solventum Intellectual Properties Company, Maplewood, MN (US)

(72) Inventors: Christopher BREACH, Poole (GB); Benjamin A. PRATT, Poole (GB); Timothy M. ROBINSON, Shillingstone (GB)

(21) Appl. No.: 18/873,131

(22) PCT Filed: May 31, 2023
(86) PCT No.: PCT/IB2023/055604

§ 371 Date: Dec. 9, 2024

Related U.S. Application Data
(60) Provisional application No. 63/351,029, filed on Jun. 10, 2022.

(51) Int. Cl. A61M 1/00; A61F 13/05

(52) U.S. Cl. CPC A61M 1/95; A61F 13/05; A61M 1/915

(57) ABSTRACT

A dressing for treating a tissue site with negative pressure includes a cover, a wound contact layer, a manifold, and at least one sensor. The cover includes a first surface and a second surface. The wound contact layer includes a first surface and a second surface where the second surface of the wound contact layer is configured to contact the tissue site. The manifold includes a first surface and a second surface. The manifold is configured to be disposed between the second surface of the cover and the first surface of the wound contact layer. The at least one sensor can include a plurality of oxygen sensing molecules dissolved in a polymer matrix. The at least one sensor is configured to change color to indicate a sensed oxygen level when the at least one sensor is acted upon by a light source.

### CROSS-REFERENCE TO RELATED APPLICATIONS

This application is a U.S. National Stage Entry of PCT International Application No. PCT/IB2023/055604, filed on May 31, 2023, which claims the benefit of priority to U.S. Provisional Application No. 63/351,029, filed on Jun. 10, 2022, which is incorporated herein by reference in its entirety.

### TECHNICAL FIELD

The invention set forth in the appended claims relates generally to tissue treatment systems and more particularly, but without limitation, to negative pressure wound therapy dressings that include in-vivo optical sensors.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for sensing changes at or near a wound site in a negative-pressure therapy environment are set forth in the appended claims. Illustrative embodiments are also provided to enable a person skilled in the art to make and use the claimed subject matter.

For example, in some embodiments, a dressing for treating a tissue site with negative pressure is described. The dressing can include a cover, a wound contact layer including at least one sensor, a first film layer, and a manifold. The cover can include a first surface and a second surface. The wound contact layer can include a first surface, a second surface, and a central aperture. The second surface of the wound contact layer can be configured to contact a periwound region of the tissue site. The first film layer can include a first surface and a second surface. The second surface of the first film layer can be configured to contact a wound of the tissue site. The manifold can include a first surface and a second surface. The manifold can be configured to be disposed between the second surface of the cover and the first surface of the first film layer. The at least one sensor can include a plurality of oxygen sensing molecules dissolved in a polymer matrix. The at least one sensor can be configured to change color to indicate a sensed oxygen level when the at least one sensor is acted upon by a light source.

In some example embodiments, the wound contact layer can further include a peripheral portion surrounding the central portion. The peripheral portion can include a plurality of apertures. In some example embodiments, the at least one sensor can be disposed within one of the plurality of apertures of the wound contact layer. In other example embodiments, the at least one sensor can be disposed between the first surface of the wound contact layer and the second surface of the cover. The at least one sensor can be offset from the plurality of apertures of the wound contact layer.

In some example embodiments, the at least one sensor can include an oxygen sensing film. In some example embodiments, the oxygen sensing film can include a low oxygen permeable polymer or a high oxygen permeable polymer. In some example embodiments, the low oxygen permeable polymer can include one of polyvinyl alcohol, ethylene vinyl alcohol, polyacrylonitrile, and polyvinylidene chloride. In some example embodiments, the high oxygen permeable polymer can include one of silicone and polyolefin elastomer.

In some example embodiments, the at least one sensor can include a multi-sensor configured to detect a fluid level, an oxygen level, and a pH level.

Also described herein is another dressing for treating a tissue site with negative pressure. The dressing can include a cover including at least one sensor, a wound contact layer, a first film layer, and a manifold. The cover can include a first surface and a second surface. The wound contact layer can include a first surface, a second surface, and a central aperture. The second surface of the wound contact layer can be configured to contact a periwound region of the tissue site. The first film layer can include a first surface and a second surface. The second surface of the first film layer can be configured to contact a wound of the tissue site. The manifold can include a first surface and a second surface. The manifold can be configured to be disposed between the second surface of the cover and the first surface of the first film layer. The at least one sensor can include a plurality of oxygen sensing molecules dissolved in a polymer matrix. The at least one sensor can be configured to change color to indicate a sensed oxygen level when the at least one sensor is acted upon by a light source.

In other example embodiments, the at least one sensor can be disposed within a recess in the first surface of the cover such that the at least one sensor is aligned with one of the plurality of apertures in the wound contact layer. In some example embodiments, the dressing may further include a recess lid removably coupled to the first surface of the cover. The recess lid may be configured to seal the at least one sensor within the recess in the first surface of the cover. In other example embodiments, the at least one sensor can be disposed on the first surface of the cover such that the sensor is aligned with one of the plurality of apertures of the wound contact layer.

In some example embodiments, the cover can further include a central portion with a central aperture and a peripheral portion. The peripheral portion of the cover can be configured to couple to the peripheral portion of the wound contact layer. In some example embodiments, the cover can further include a second film layer with a first surface and a second surface. The second film layer can be configured to substantially alight with the central aperture of the cover and the second surface of the second film layer can couple to the first surface of the manifold. In some example embodiments, the at least one sensor can be disposed within a recess in the first surface of the second film layer. In some example embodiments, the dressing can further include a recess lid removably coupled to the first surface of the second film layer. The recess lid can be configured to seal the at least one sensor within the recess in the first surface of the second film layer. In some embodiments, the at least one sensor can be disposed between the first surface of the manifold and the second surface of the second film layer. In other embodiments, the dressing can further include at least one sealed cavity between the first surface of the manifold and the second surface of the second film layer. The at least one sensor can be disposed within the at least one sealed cavity.

Also described herein is another dressing for treating a tissue site with negative pressure. The dressing can include a cover, a wound contact layer, a first film layer, a manifold, and at least one sensor. The cover can include a first surface and a second surface. The wound contact layer can include a first surface, a second surface, and a central aperture. The second surface of the wound contact layer can be configured to contact a periwound region of the tissue site. The first film layer can include a first surface and a second surface. The second surface of the first film layer can be configured to contact a wound of the tissue site. The manifold can include a first surface and a second surface. The manifold can be configured to be disposed between the second surface of the cover and the first surface of the first film layer. The at least one sensor can be recessed into the second surface of the first film layer. The at least one sensor can include a plurality of oxygen sensing molecules dissolved in a polymer matrix. The at least one sensor can be configured to change color to indicate a sensed oxygen level when the at least one sensor is acted upon by a light source.

Also described herein is another dressing for treating a tissue site with negative pressure. The dressing can include a cover, a wound contact layer, a first film layer, a manifold, and at least one sensor. The cover can include a first surface and a second surface. The wound contact layer can include a first surface, a second surface, and a central aperture. The second surface of the wound contact layer can be configured to contact a periwound region of the tissue site. The first film layer can include a first surface and a second surface. The second surface of the first film layer can be configured to contact a wound of the tissue site. The manifold can include a first surface and a second surface. The manifold can be configured to be disposed between the second surface of the cover and the first surface of the first film layer. The at least one sensor can be disposed between the second surface of the manifold and the first surface of the first film layer. The at least one sensor can include a plurality of oxygen sensing molecules dissolved in a polymer matrix. The at least one sensor can be configured to change color to indicate a sensed oxygen level when the at least one sensor is acted upon by a light source.

In some example embodiments, the at least one sensor can be disposed proximate a window of the manifold such that the at least one sensor is visible through the window of the manifold.

Also described herein is a method of treating a tissue site with negative pressure. The method can include disposing a dressing on the tissue site, applying negative pressure, from a negative pressure source, to the dressing, and monitoring the dressing for changes in a sensed parameter at the tissue site. The dressing can include a cover, a wound contact layer, a manifold, and at least one sensor. The cover can include a first surface and a second surface. The wound contact layer can include a first surface and a second surface. The second surface of the wound contact layer can be configured to contact the tissue site. The manifold can include a first surface and a second surface. The manifold can be configured to be disposed between the second surface of the cover and the first surface of the wound contact layer. The at least one sensor can include a plurality of parameter sensing molecules dissolved in a polymer matrix. The at least one sensor can be configured indicate the sensed parameter when the at least one sensor is acted upon by a light source.

In some example embodiments, monitoring the dressing for changes in the sensed parameter at the tissue site can include detecting a change of color in that at least one sensor when the at least one sensor is acted upon by a light source.

In some example embodiments, the sensed parameter can include at least one of a fluid level, an oxygen level, and a pH.

Also described herein is a wound contact layer for treating a tissue site. The wound contact layer can include a central portion, a peripheral portion and at least one sensor. The peripheral portion can surround the central portion in some example embodiments. In some example embodiments, the peripheral portion can include a plurality of apertures. In some example embodiments, the at least one sensor can include a plurality of oxygen sensing molecules dissolved in a polymer matrix. The at least one sensor can be integrated into the peripheral portion separate from each of the plurality of apertures. In some example embodiments, the at least one sensor can be configured to change color to indicate a sensed oxygen level when the at least one sensor is acted upon by a light source.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2 is an exploded view of an example of a dressing of FIG. 1, illustrating additional details that may be associated with some embodiments;

FIG. 3 is a perspective view of the dressing of FIG. 2, as assembled;

FIG. 4 is a cross sectional view of the dressing of FIG. 3 taken at line 4-4, applied to an example tissue site, and illustrating additional details associated with some examples of the therapy system of FIG. 1;

FIG. 5A is a perspective view of another example of the dressing of FIG. 1, illustrating additional details that may be associated with some embodiments;

FIG. 5B is a perspective view of the dressing of 5A including sensor covers that may be associated with some example embodiments;

FIG. 5C is a cross sectional view of the dressing of FIG. 5B taken at line 5C-5C;

FIG. 5D is a cross sectional view of the dressing of FIG. 5B taken at line 5D-5D;

FIG. 6A is a cross sectional view of a sensor disposed within a wound contact layer of the dressing of FIG. 1;

FIG. 6B is a cross sectional view of the sensor integrated into the wound contact layer of the dressing of FIG. 1;

FIG. 6C is a cross sectional view of the sensor disposed between the wound contact layer and a cover of the dressing of FIG. 1;

FIG. 6D is a cross sectional view of the sensor disposed at another location between the wound contact layer and a cover of the dressing of FIG. 1;

FIG. 6E is a cross sectional view of the sensor disposed within a recess of the cover of the dressing of FIG. 1;

FIG. 6F is a cross sectional view of the sensor disposed on the cover opposite the wound contact layer of the dressing of FIG. 1;

FIG. 7A is a cross sectional view of the sensor disposed on the wound contact layer opposite a manifold of the dressing of FIG. 1;

FIG. 7B is a cross sectional view of the sensor disposed between the manifold and the wound contact layer of the dressing of FIG. 1;

FIG. 7C is a cross sectional view of the sensor disposed between the manifold and the cover of the dressing of FIG. 1;

FIG. 7D is a cross sectional view of the sensor disposed at another location between the manifold and the cover of the dressing of FIG. 1;

FIG. 7E is a cross sectional view of the sensor disposed within a sealed chamber between the manifold and the cover of the dressing of FIG. 1;

FIG. 7F is a cross sectional view of the sensor disposed on the cover opposite the manifold of the dressing of FIG. 1;

FIG. 8A is a cross sectional view of the sensor disposed within a recess of the cover of the dressing of FIG. 1;

FIG. 8B is a cross sectional view of the sensor disposed within another recess of the cover of the dressing of FIG. 1;

FIG. 9 is a side view of a multi-sensor that can be used with the dressing of FIG. 1; and

FIG. 10 is a perspective view of the dressing of FIG. 5A deployed at a tissue site being acted upon by a light source.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

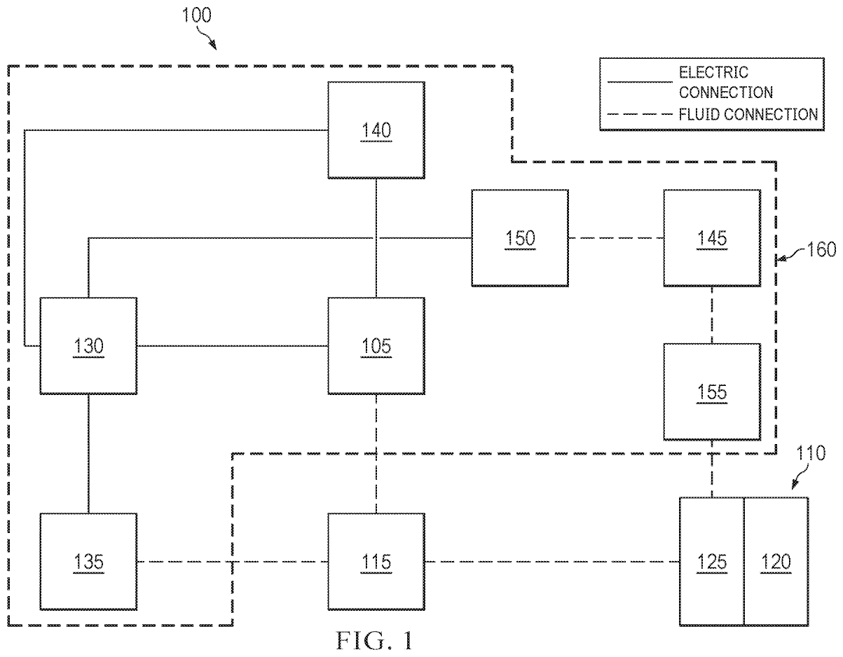

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g., saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit 160.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or a computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be an apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, to a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source.

In some example embodiments, the cover 125 may be a polymer drape, such as a polyurethane film, that is permeable to water vapor but impermeable to liquid. In other embodiments, the cover 125 may be impermeable to both water vapor and liquids. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and INSPIRE® 2301 and INSPIRE® 2327 polyurethane films, commercially available from Exopack Advanced Coatings, Wrexham, United Kingdom. In some embodiments, the cover 125 may comprise INSPIRE® 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. In some example embodiments, the controller 130 can operate the negative-pressure source 105 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of +25 mmHg/min. and a descent rate set at −25 mmHg/min. In other embodiments of the therapy system 100, the triangular waveform may vary between negative pressure of 25 and 135 mmHg with a rise rate of about +30 mmHg/min and a descent rate or about −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second and 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 150 to move solution from the solution source 145 to the tissue interface 120. Additionally or alternatively, the solution source 145 may be elevated to a height sufficient to allow gravity to move solution into the tissue interface 120.

The controller 130 may also control the fluid dynamics of instillation at 425 by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 120, or it may be implemented to provide a dynamic pressure mode of operation to vary the flow rate of instillation solution through the tissue interface 120. Alternatively, the application of negative pressure may be implemented to provide an intermittent mode of operation to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied. The controller 130 may be utilized to select a mode of operation and the duration of the negative pressure treatment before commencing another instillation cycle by instilling more solution.

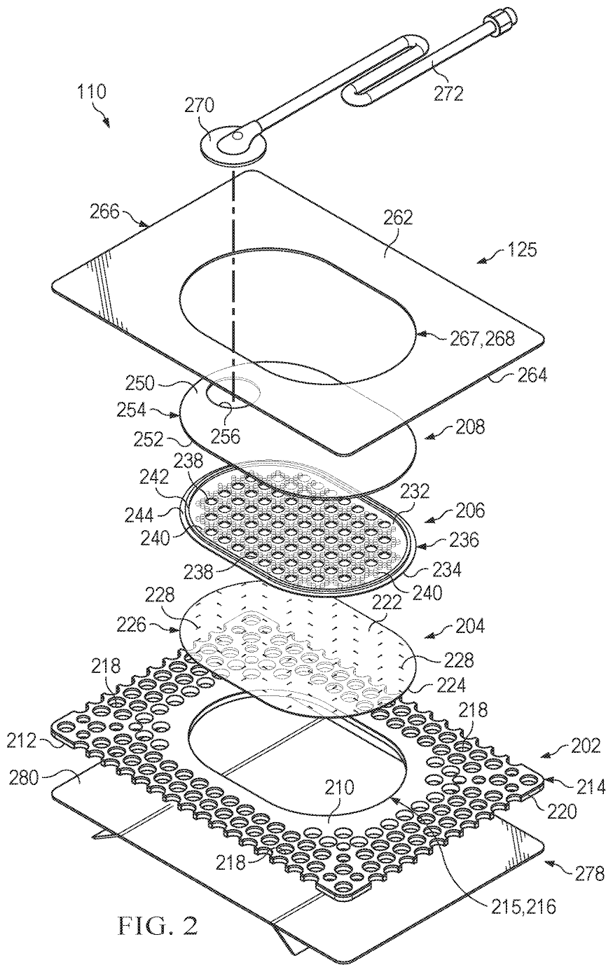

FIG. 2 is an exploded view of an example of the dressing 110 of FIG. 1, illustrating additional details that may be associated with some embodiments. The dressing 110 may include a sealing layer or a wound contact layer 202, a first film layer 204, a manifold layer or a manifold 206, a second film layer 208, and a drape or the cover 125. The wound contact layer 202 may be formed from a soft, pliable material suitable for providing a fluid seal with a tissue site, such as a suitable gel material, and may have a substantially flat surface. In some embodiments, the wound contact layer 202 may include, without limitation, a silicone gel, a soft silicone, hydrocolloid, hydrogel, polyurethane gel, polyolefin gel, hydrogenated styrenic copolymer gel, a foamed gel, soft closed-cell foams such as polyurethanes and polyolefins coated with adhesives, polyurethane, polyolefin, or hydrogenated styrenic copolymers. In some embodiments, the wound contact layer 202 may have a thickness in a range of about 200 micrometers to about 1,000 micrometers. In some embodiments, the wound contact layer 202 may be formed from hydrophobic or hydrophilic materials.

In some embodiments, the wound contact layer 202 may be include or be formed from a hydrophobic or hydrophobic-coated material. For example, the wound contact layer 202 may be formed by coating a spaced material, such as woven, nonwoven, molded, or extruded mesh, with a hydrophobic material such as a soft silicone.

The wound contact layer 202 may have a first surface 210 and a second surface 212 opposite the first surface 210. The wound contact layer 202 may also include a peripheral portion or a periphery 214 defined by an outer perimeter of the wound contact layer 202, and central portion 215 containing a treatment aperture 216 formed through the wound contact layer 202. In some embodiments, the treatment aperture 216 may have an outline complementary to or corresponding to an outer perimeter of the manifold 206. The wound contact layer 202 may also include a plurality of apertures 218 formed through the wound contact layer 202. In some embodiments, the plurality of apertures 218 may be formed through a region of the wound contact layer 202 between the treatment aperture 216 and the periphery 214.

In some embodiments, the periphery 214 of the wound contact layer 202 may be or may include a sensor 220. The sensor 220 may be configured to change color to indicate a sensed parameter when the sensor 220 is acted upon by a light source. For example, in some embodiments, the sensor 220 may be configured to sense an oxygen level. When acted upon by a light source, the sensor 220 may changing color. In some embodiments, the intensity of the color may be dependent on the sensed parameter such as the sensed oxygen level. For example, the sensor 220 may emit an intense light or an intense color when there is a relatively low sensed oxygen level and the sensor 220 may emit a relatively dull or dim light or color when there is a relatively high sensed oxygen level. In some embodiments, the sensor 220 may emit a base level color when exposed to the ambient environment. Thus, when the sensor 220 senses an oxygen level less than the oxygen level of the ambient environment, the sensor 220 may emit a color that is more intense or more vibrant than the base level color. When the sensor 220 senses an oxygen level that is greater than the oxygen level of the ambient environment, the sensor 220 may emit a color that is less intense or vibrant than the base level color. In other embodiments, the sensor 220 may emit light differently than described above but may still change when exposed to a light source such that a sensed parameter is displayed by the sensor 220. In some embodiments, the sensor 220 may be configured to sense other parameters such as a wound temperature, a pH level, or a presence of fluid. In embodiments where the sensor 220 is configured to sense a pH level, the sensor 220 may function substantially as described above with respect to sensing oxygen. For example, the intensity of the emitted light may be dependent on the sensed pH level. In embodiments where the sensor 220 is configured to detect the presence of fluid, the sensor 220 may be partially or fully opaque when dry and may be transparent when exposed to fluid. When the sensor 220 transitions from opaque to transparent, the sensor 220 has detected the presence of fluid.

In some embodiments, the sensor 220 may be a dye that may be integrated or blended into the dressing 110. For example, the sensor 220 may be integrated into the periphery 214 of the wound contact layer 202 in some embodiments. In other embodiments, the sensor 220 may be a film that contains luminescent materials such as transition metal ligands or chemical sensing materials. The chemical sensing materials can be oxygen sensing materials or molecules or pH sensing materials or molecules in some embodiments. The film that may be coupled to or deposited onto a layer of the dressing 110 such as the wound contact layer 202. In some embodiments, the sensor 220 may be an oxygen sensing film. The oxygen sensing film may include a polymer matrix with oxygen sensing molecules dissolved in the polymer matrix. Biocompatible silicones and biocompatible oxygen sensing materials may be mixed or blended with transparent medical-grade polymers to create the desired oxygen sensing film. In some embodiments, the oxygen sensing molecules may be porphyrins. The oxygen sensing film can include either a low oxygen permeable polymer or a high oxygen permeable polymer. The low oxygen permeable polymer can include at least one of polyvinyl alcohol, ethylene vinyl alcohol, polyacrylonitrile, polyvinylidene chloride, or another similar material. The high oxygen permeable polymer can include at least one of silicone, polyolefin elastomer, or another similar material. In other embodiments, the sensor 220 may be a biocompatible oxygen sensing material such as porphyrins as described above printed directly onto a layer of the dressing 110 such as the wound contact layer 202.

As shown in FIG. 2, the sensor 220 is deposited onto the second surface 212 of the wound contact layer 202. In some embodiments, the sensor 220 may extend around the periphery 214 of the wound contact layer 202 and in other embodiments, the sensor 220 may be deposited onto the entirety of the second surface 212 of the wound contact layer 202. In still other embodiments, the sensor 220 may be deposited at discrete locations on the second surface 212 of the wound contact layer 202. The sensor 220 may be at any location of the second surface 212 of the wound contact layer 202 that will be disposed proximate to a periwound region of a tissue site when the dressing 110 is disposed on a tissue site. When the dressing 110 is disposed at the tissue site, the sensor 220 may be configured to sense a parameter in a periwound region of a wound where the wound contact layer 202 is coupled to a tissue site. For example, the sensor 220 may be configured to sense an oxygen level at a periwound region of a tissue site when the dressing 110 is coupled to the tissue site.

In some embodiments, the plurality of apertures 218 may be formed by cutting, perforating, or applying local radio-frequency or ultrasonic energy through the wound contact layer 202. In some embodiments, the plurality of apertures 218 may be formed by other suitable techniques for forming an opening or perforation in the wound contact layer 202. In some embodiments, the plurality of apertures 218 may have a uniform distribution pattern. In other embodiments, the plurality of apertures 218 may be randomly distributed. In some embodiments, the plurality of apertures 218 may have many any combination of shapes, including circles, squares, stars, ovals, polygons, slits, complex curves, rectilinear shapes, or triangles.

In some embodiments, each of the plurality of apertures 218 may have uniform or similar geometric properties. For example, each of the plurality of apertures 218 may be a circular aperture and have substantially the same diameter. In some embodiments, each of the plurality of apertures 218 may have a diameter in a range of between about 1 millimeter and about 20 millimeters.

In some embodiments, the geometric properties of the plurality of apertures 218 may vary. For example, the diameters of the plurality of apertures 218 may vary depending on the positioning of the apertures 218 in the wound contact layer 202. In some embodiments, at least some of the plurality of apertures 218 may have a diameter in a range of between about 5 millimeters to about 10 millimeters. In some embodiments, at least some of the plurality of apertures 218 may have a diameter in a range of between about 7 millimeters and about 9 millimeters. In some embodiments, the wound contact layer 202 may include corners, and the plurality of apertures 218 disposed at or near the corners may have diameters in a range of between about 7 millimeters and about 8 millimeters.

In some embodiments, at least some of the plurality of apertures 218 positioned proximate the periphery 214 may have an interior that is cut open or exposed at the periphery 214 and is in lateral communication in a lateral direction (relative to the first surface 210 and/or the second surface 212) with the periphery 214. In some embodiments, the lateral direction may refer to a direction in a same plane as the first surface 210 and/or the second surface 212 and extending towards the periphery 214. In some embodiments, at least some of the plurality of apertures 218 positioned proximate to or at the periphery 214 may be spaced substantially equidistantly around the periphery 214. Alternatively, in some embodiments, the spacing of the plurality of apertures 218 proximate to or at the periphery 214 may be spaced irregularly.

The first film layer 204 may include a suitable structure for controlling or managing fluid flow. In some embodiments, the first film layer 204 may be a fluid-control layer that includes a liquid-impermeable, vapor-permeable elastomeric material. In some embodiments, the first film layer 204 may be formed from or include a polymer film. For example, in some embodiments, the first film layer 204 may be formed from or include a polyolefin film, such as a polyethylene film. In some embodiments, the first film layer 204 may be substantially clear or optically transparent. In some embodiments, the first film layer 204 may be formed from or include the same material as the cover 125. In some embodiments, the first film layer 204 may be formed from or include a biocompatible polyurethane film tested and certified according to the USP Class VI Standard. In some embodiments, the first film layer 204 may also have a smooth or matte surface texture. In some embodiments, the first film layer 204 may have a glossy or shiny finish equal to or exceeding a grade B3 according to the Society of Plastics Industry (SPI) standards. In some embodiments, the surface of the first film layer 204 may be a substantially flat surface, with height variations in a range of about 0.2 millimeters to about 1 centimeter.

In some embodiments, the first film layer 204 may be hydrophobic. The hydrophobicity of the first film layer 204 may vary, but may have a contact angle with water of at least 90 degrees in some examples. In some embodiments, the first film layer 204 may have a contact angle with water of no more than 150 degrees. In some embodiments, the first film layer 204 may have a contact angle with water in a range of about 90 degrees to about 120 degrees, or in a range of about 120 degrees to about 150 degrees. Water contact angle may be measured using any standard apparatus. Although manual goniometers may be used to visually approximate contact angles, contact angle measuring instruments may often involve integrated systems that include a level stage, a liquid dropper (such as a syringe), a camera, and software designed to calculated contact angles more accurately and precisely. Non-limiting examples of such integrated systems include the FTÅ125, FTÅ200, FTÅ2000, and FTÅ4000 systems, all commercially available from First Ten Angstroms, Inc., of Portsmouth, Virginia, and the DTA25, DTA30, and DTA100 systems, all commercially available from Kruss GmbH of Hamburg, Germany. Unless otherwise specified, water contact angles herein are measured using deionized and/or distilled water on a level sample surface for a sessile drop added from a height of no more than five centimeters in air at 20-25° C. and 20-50% relative humidity. Contact angles herein represent averages of five to nine measured values, with the highest and lowest measure values discarded. In some embodiments, the hydrophobicity of the first film layer 204 may be further enhanced with a hydrophobic coating of other materials, such as silicones and fluorocarbons.

The first film layer 204 may also be suitable for welding to other layers, including the manifold 206 and the second film layer 208. In some embodiments, the first film layer 204 may be adapted for welding to polymers such as polyurethane, polyurethane films, and polyurethane foams using heat welding, radio-frequency (RF) welding, ultrasonic welding, or other methods. RF welding may be particularly suitable for more polar materials, such as polyurethane, polyamides, polyesters, and acrylates. Sacrificial polar interfaces may be used to facilitate RF welding of less polar film materials, such as polyethylene.

The area density of the first film layer 204 may vary according to a prescribed therapy or application. In some embodiments, an area density of less than 40 grams per square meter may be suitable. In some embodiments, the area density of the first film layer 204 may be in a range of about 20 grams per square meter to about 30 grams per square meter.

In some embodiments, the first film layer 204 may be formed from or include a hydrophobic polymer, such as a polyethylene film. The simple and inert structure of polyethylene provides a surface that interacts little, if any, with biological tissues and fluids, and provides a surface that may encourage the free flow of liquids and exhibits a low adherence to tissues and fluids, properties that may be particularly advantageous for many applications. In some embodiments, the first film layer 204 may be formed from other polymeric films such as polyurethanes, acrylics, polyolefins (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate styreneics, silicones, fluoropolymers, and acetates. In some embodiments, the first film layer 204 may have a thickness in a range of about 20 micrometers to about 500 micrometers. In some embodiments, the first film layer 204 may have a thickness of about 23 micrometers, about 25 micrometers, about 100 micrometers, about 250 micrometers, about 300 micrometers, and about 500 micrometers. In some embodiments, the first film layer 204 may include a polar film suitable for lamination to the polyethylene film, such as polyamides, co-polyesters, ionomers, and acrylics. In some embodiments, the first film layer 204 may include a tie layer to improve the bond between the polyethylene and polar film layers. In some embodiments, the tie layer may include ethylene vinyl acetate or modified polyurethanes. In some embodiments, the first film layer 204 may include an ethyl methyl acrylate (EMA) film.

The first film layer 204 may have a first surface 222 and a second surface 224 opposite the first surface 222. The first film layer 204 may further include a periphery 226 defined by an outer perimeter of the first film layer 204. In some embodiments, the periphery 226 may be a stadium, disco rectangular, or obround shape. The first film layer 204 may also include one or more fluid passages 228 formed through the first film layer 204, and which may be distributed uniformly or randomly across the first film layer 204.

In some embodiments, the fluid passages 228 may function as bi-directional and fluid-responsive valves. For example, each fluid passage 228 may be an elastic passage that is normally unstrained to prevent or substantially reduce fluid flow across the fluid passage 228 and can expand or open to allow fluid flow across the fluid passage 228 in response to a pressure gradient applied across the fluid passage 228. In some embodiments, the fluid passages 228 may include perforations formed in the first film layer 204. Perforations may be formed by removing material from the first film layer 204 or cutting through the first film layer 204. In some embodiments, cutting through the first film layer 204 may deform the edges of the perforations. In some embodiments, the fluid passages 228 may be sufficiently narrow to form a seal or a fluid restriction to substantially reduce or prevent fluid flow across the fluid passage 228, particularly in the absence of a pressure differential. In some embodiments, one or more of the fluid passages 228 may be an elastomeric valve that is normally closed when unstrained to prevent liquid flow across the valve, and that can open in response to a pressure gradient. In some embodiments, the fluid passages 228 may include fenestrations formed through the first film layer 204. Fenestrations may be formed by removing material from the first film layer 204, but the amount of material removed and the resulting dimensions of the fenestrations may be up to an order of magnitude less than perforations, and may not deform the edges.

In some embodiments, the fluid passages 228 may include one or more slits, slots, or combinations of slits and slots in the first film layer 204. In some embodiments, the fluid passages 228 may include linear slots having a length less than about five millimeters and a width less than about two millimeters. In some embodiments, the length may be at least about two millimeters, and the width may be at least about 0.5 millimeters. In some embodiments, the length may be in a range of about two millimeters to about five millimeters and the width may be in a range of about 0.5 millimeters to about two millimeters, with a tolerance of about 0.1 millimeters. In some embodiments, the length may be about three millimeters. Such dimensions and tolerances may be achieved with a laser cutter, for example. In some embodiments, slots of such configurations may function as imperfect valves that substantially reduce liquid flow in a normally closed or resting state. Such slots may form a flow restriction without being completely closed or sealed. The slots can expand or open wider in response to a pressure gradient applied across the slot to allow increased liquid flow through the slot.

In some embodiments, the fluid passages 228 may include linear slits having a length of less than about five millimeters. In some embodiments, the length of the linear slits may be at least about two millimeters. In some embodiments, the length of the linear slits may be in a range of about two millimeters to about five millimeters, with a tolerance of about 0.1 millimeters. In some embodiments, the length of the linear slits may be about three millimeters.

In some embodiments, not pictured herein, the first film layer 204 may be integral with the wound contact layer 202. For example, the wound contact layer 202 may not contain the treatment aperture 216 and may be a continuous layer. The central portion 215 of the wound contact layer 202 may include the fluid passages 228 of the first film layer in some embodiments.

In some embodiments, the manifold 206 may be formed as a substantially sheet-like structure having a first surface 232 and a second surface 234 opposite the first surface 232. In some embodiments, the manifold 206 may further include a periphery 236 defined by an outer perimeter of the manifold 206. In some embodiments, the periphery 236 of the manifold 206 may be substantially similar to or coextensive with the periphery 226 of the first film layer 204. In some embodiments, the manifold 206 may be formed from a sheet of polyurethane, such as a vacuum-formed sheet of polyurethane having a thickness of about 0.5 millimeters. In some embodiments, the manifold 206 may be formed from a polymer material that is substantially clear or optically transparent, allowing the user to see through the manifold 206.

In some embodiments, a plurality of windows 238 may be removed from the manifold 206 and form a grid pattern. For example, the plurality of windows 238 may be arranged in a pattern of rows and columns. The center of each window 238 of the plurality of windows 238 may be aligned with the center of each other window 238 of the plurality of windows 238 within a row, and the center of each window 238 of the plurality of windows 238 may be aligned with the center of each other window 238 of the plurality of windows 238 within a column. In some embodiments, a plurality of standoffs 240 may be formed on the second surface 234 of the manifold 206. In some embodiments, the plurality of standoffs 240 may form a grid pattern. For example, the plurality of standoffs 240 may be arranged in a pattern of rows and columns. The center of each standoff 240 of the plurality of standoffs 240 may be aligned with the center of each other standoff 240 of the plurality of standoffs 240 within a row, and the center of each standoff 240 of the plurality of standoffs 240 may be aligned with the center of each other standoff 240 of the plurality of standoffs 240 within a column. In some embodiments, each row of the plurality of windows 238 may be disposed adjacent to a row of the plurality of standoffs 240, and each column of the plurality of windows 238 may be disposed adjacent to a column of the plurality of standoffs 240. In some embodiments, the plurality of windows 238 and the plurality of standoffs 240 may be arranged in a pattern such that rows of the pattern alternate between rows of the plurality of windows 238 and rows of the plurality of standoffs 240, and columns of the pattern alternate between columns of the plurality of windows 238 and columns of the plurality of standoffs 240.

In some embodiments, each window 238 may be substantially circular in profile in the plane of the first surface 232 of the manifold 206. In some embodiments, each standoff 240 may be substantially circular in profile and protrude outwardly in a substantially orthogonal manner from the plane of the second surface 234 of the manifold 206. In some embodiments, a diameter of each window 238 of the plurality of windows 238 may be greater than a diameter of each standoff 240 of the plurality of standoffs 240. For example, each window 238 of the plurality of windows 238 may have a diameter of about eight millimeters, and each standoff 240 of the plurality of standoffs 240 may have a diameter of about three millimeters. In some embodiments, each standoff 240 of the plurality of standoffs 240 may have a height of in a range of about 0.5 millimeters to about 3 millimeters. In some embodiments, each standoff 240 of the plurality of standoffs 240 may have a height of about 2.5 millimeters. In some embodiments, each standoff 240 of the plurality of standoffs 240 may have a height of about 3 millimeters. In some embodiments, each standoff 240 of the plurality of standoffs 240 within a row may be spaced a distance of about four millimeters on center from an adjacent standoff 240 of the plurality of standoffs 240 within a row, and each standoff 240 of the plurality of standoffs 240 within a column may be spaced a distance of about four millimeters on center from an adjacent standoff 240 of the plurality of standoffs 240 within a column. In some embodiments, the plurality of standoffs 240 may be right cylinders with hemispherical ends, such as half-capsules, and may be formed on and protrude substantially away from the second surface 234 of the manifold 206 in a direction substantially normal to the second surface 234. In some embodiments, each of the plurality of standoffs 240 may have a height in a range of about 2.5 millimeters to about three millimeters.

In some embodiments, the manifold 206 may include a raised portion, such as a lip portion or a boss 242. In some embodiments, the boss 242 may protrude outwardly in a substantially orthogonal manner from the plane of the first surface 232 of the manifold 206. In some embodiments, the boss 242 may have a scaled down profile or outline similar to the shape of the periphery 236 of the manifold 206. In some embodiments, the manifold 206 may also have a border region 244 between the boss 242 and the periphery 236 of the manifold. In some embodiments, the border region 244 may not include any of the windows 238 of the plurality of windows 238 or of the standoffs 240 of the plurality of standoffs 240.

The second film layer 208 may have a first surface 250 and a second surface 252 opposite the first surface 250. The second film layer 208 may further include a periphery 254 defined by a perimeter of the second film layer 208. A negative-pressure aperture, such as aperture 256 may be formed through the second film layer 208. In some embodiments, the second film layer 208 may be formed from or include any of the materials previously described with respect to the cover 125 and/or the first film layer 204.

The cover 125 may include a first surface 262 and a second surface 264 opposite the first surface 262. The cover 125 may further include a peripheral portion or a periphery 266 defined by an outer perimeter of the cover 125. In some embodiments, the cover may have a central portion 267 with a central aperture 268 formed through the cover 125. In some embodiments the cover 125 may act as a barrier between the ambient environment and the sensor 220. For example, the cover 125 may be substantially oxygen impermeable to block any oxygen from reaching the sensor 220 and impacting a sensed oxygen level at a periwound region of a tissue site.

In some embodiments, not pictured herein, the second film layer 208 and the central aperture 268 of the cover 125 may be omitted and the cover 125 may be a continuous layer such that the second surface 264 of the cover 125 contacts the first surface 232 of the manifold 206. In other embodiments, the second film layer 208 may be a portion of the cover 125 and may align with the central aperture 268 of the cover 125 to create a barrier between the ambient environment and the remainder of the dressing 110.

In some embodiments, the periphery 214 of the wound contact layer 202 may be substantially coextensive with the periphery 266 of the cover 125. In some embodiments, the periphery 226 of the first film layer 204, the periphery 236 of the manifold 206, and the periphery 254 of the second film layer 208 may be substantially coextensive. In some embodiments, the outline of the treatment aperture 216 of the wound contact layer 202 may be substantially coextensive with the outline of the central aperture 268 of the cover 125. In some embodiments, the outlines of the treatment aperture 216 and the central aperture 268 may be substantially similar to the outlines of the periphery 226 of the first film layer 204, the periphery 236 of the manifold 206, and the periphery 254 of the second film layer 208. In some embodiments, the outlines of the treatment aperture 216 and the central aperture 268 may be substantially similar to but scaled down from the outlines of the periphery 226 of the first film layer 204, the periphery 236 of the manifold 206, and the periphery 254 of the second film layer 208. In assembled form, the wound contact layer 202, the first film layer 204, the manifold 206, the second film layer 208, and the cover 125 may be stacked such that the periphery 214 of the wound contact layer 202 is aligned with the periphery 266 of the cover 125, and the periphery 226 of the first film layer 204 is aligned with the periphery 236 of the manifold 206, and the periphery 254 of the second film layer 208. In some embodiments, the treatment aperture 216 may be aligned with the central aperture 268, and the periphery 226 of the first film layer 204, the periphery 236 of the manifold 206, and the periphery 254 of the second film layer 208 may be positioned such that they are aligned with and evenly extend past the outlines of the treatment aperture 216 and the central aperture 268.

In some embodiments, a portion of the first surface 210 of the wound contact layer 202 around the treatment aperture 216 may be coupled to a portion of the second surface 224 of the first film layer 204 near the periphery 226, and a portion of the second surface 264 of the cover 125 around the central aperture 268 may be coupled to a portion of the first surface 250 of the second film layer 208 near the periphery 254. In some embodiments, a portion of the first surface 210 of the wound contact layer 202 between the periphery 214 and the treatment aperture 216 may be coupled to a portion of the second surface 264 of the cover 125 between the periphery 266 and the central aperture 268.

Some examples of the dressing 110 may also include a dressing interface 270 and a fluid conductor 272. In various implementations, the fluid conductor 272 may be a flexible tube that can be fluidly coupled on one end to the dressing interface 270. In various implementations, the dressing interface 270 may be an elbow connector that can be placed over aperture 256 to provide a fluid path between the fluid conductor 272 and the interior of the dressing 110.

In some embodiments, the dressing 110 may further include a release liner 278 to protect the wound contact layer 202 and the adhesive coated on the second surface 264 of the cover 125 prior to use. The release liner 278 may also provide stiffness to assist with, for example, deployment of the dressing 110. In various implementations, the release liner may include a polyethylene terephthalate (PET) or similar polar semi-crystalline polymer. The use of a polar semi-crystalline polymer for the release liner 278 may substantially preclude wrinkling or other deformation of the dressing 110. The polar semi-crystalline polymer may be highly orientated and resistant to softening, swelling, or other deformation that may occur when objects are brought into contact with the layers and/or components of the dressing 110, or when the dressing 110 is subjected to temperature or environmental variations, or during sterilization. Further, a release agent may be disposed on a first surface 280 of the release liner 278 that is configured to contact the second surface 212 of the wound contact layer 202 and the adhesive disposed on the second surface 264 of the cover 125. For example, the release agent may be a silicone coating and may have a release factor suitable to facilitate removal of the release liner 278 by hand and without damaging or deforming the dressing 110. In various implementations, the release agent may be a fluorocarbon or a fluorosilicone. In various implementations, the release liner 278 may be uncoated or otherwise used without a release agent.

FIG. 3 is a perspective view of an assembled example of the dressing 110 of FIG. 2. In some example embodiments, the cover 125, the second film layer 208, the manifold 206, the first film layer 204, and/or the wound contact layer 202 may be substantially clear or optically transparent, allowing for visualization of the layers of the dressing 110 as well as visualization through the windows 238 of the manifold 206. The sensor 220 may also be visible through the cover 125 in some embodiments such that light or color may be observed through the cover 125 and the wound contact layer 202 when the dressing 110 is acted upon by a light source.

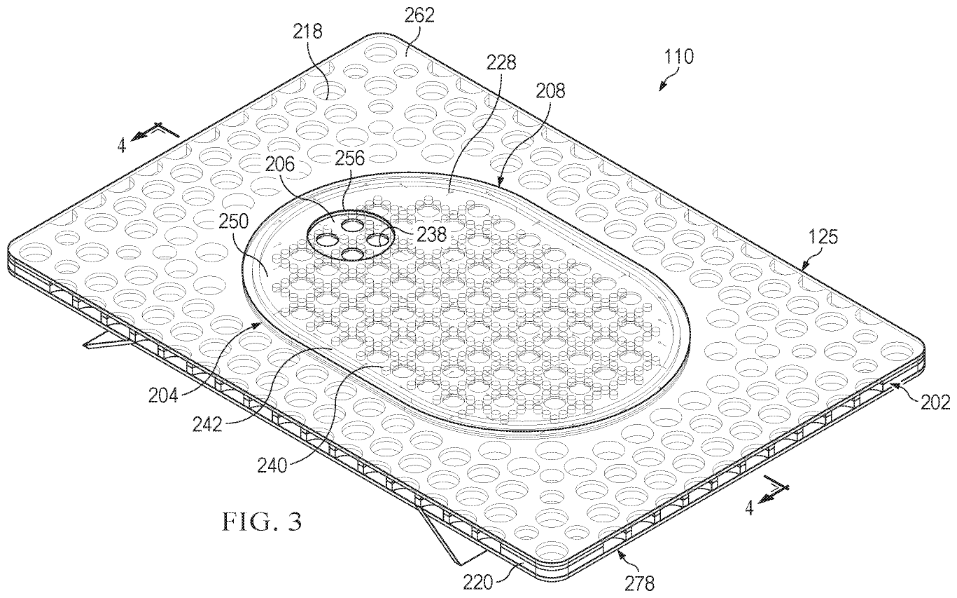

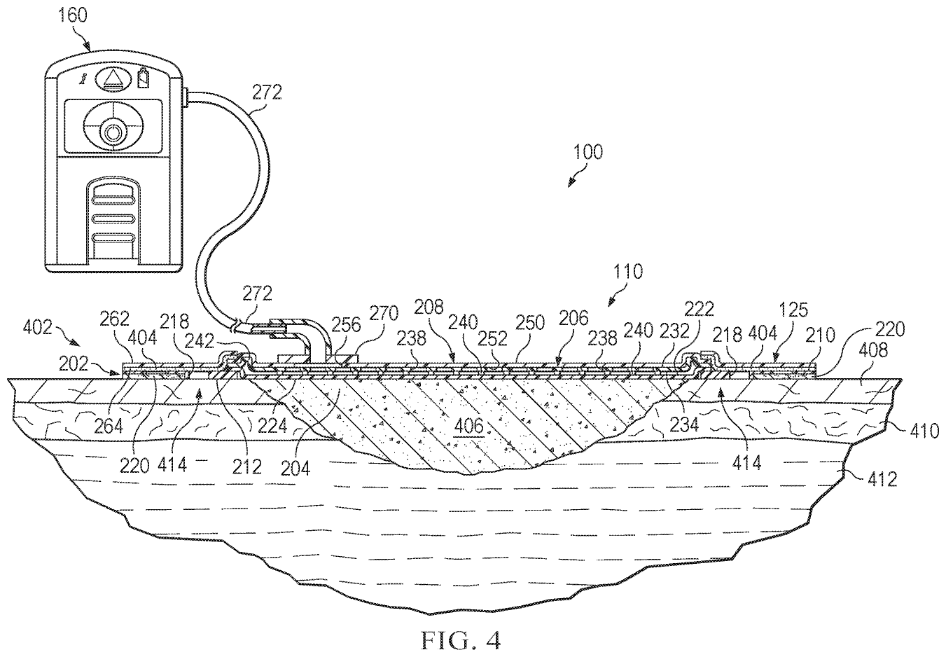

FIG. 4 is a cross sectional view illustrating the dressing 110 of FIG. 3, taken at line 4-4, applied to a tissue site 402, and illustrating additional details associated with the therapy system 100 of FIG. 1. In some embodiments, the second surface 264 of the cover 125 may be coated with an adhesive layer 404, and at least a portion of the cover 125 may be coupled to at least a portion of the first surface 210 of the wound contact layer 202 with the adhesive layer 404. The adhesive layer 404 may be any of the attachment devices previously discussed with reference to FIG. 1. In some embodiments, a portion of the second surface 264 of the cover 125 may be coupled to a portion of the first surface 250 of the second film layer 208, for example, at the periphery 254, by the adhesive layer 404.

In some applications, the dressing 110 may be applied to the tissue site 402 to cover a wound 406. The tissue site 402 may be or may include a defect or targeted treatment site, such as the wound 406, which may be partially or completely filled or covered by the dressing 110. In some examples, the wound 406 may be in an epidermis 408. In some examples, the wound 406 may extend through the epidermis 408 and into a dermis 410. In other examples, the wound 406 may extend through the epidermis 408 and the dermis 410 into a subcutaneous tissue 412. In some embodiments, at least a portion of the second surface 212 of the wound contact layer 202 may be brought into contact with a portion of the epidermis 408 surrounding the wound 406, such as a periwound region 414. At least a portion of the second surface 224 of the first film layer 204 may be placed within, over, on, against, or otherwise proximate to the wound 406.

In operation, negative pressure may be provided to the wound 406, and/or fluid may be removed from the wound 406 by the negative-pressure source 105 of the therapy unit 160 through the dressing interface 270 and the fluid conductor 272. While the dressing 110 is applied at the tissue site 402, the sensor 220 may be in contact with the periwound region 414. The sensor 220 may be configured to detect a parameter at the periwound region 414. For example, the sensor 220 may be configured to detect an oxygen level at the periwound region 414. In other embodiments, the sensor 220 may be configured to detect another parameter such as a pH or a fluid level at the periwound region 414.

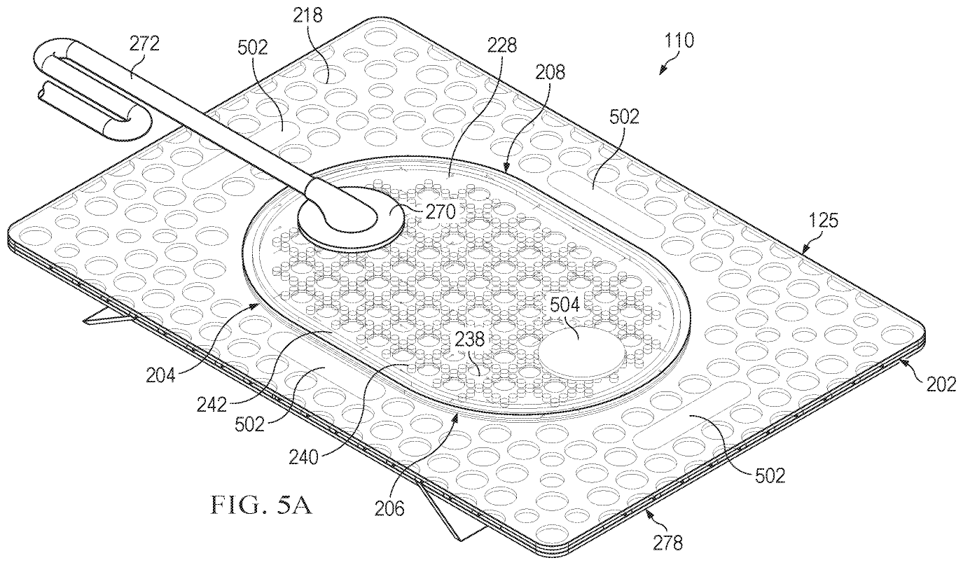

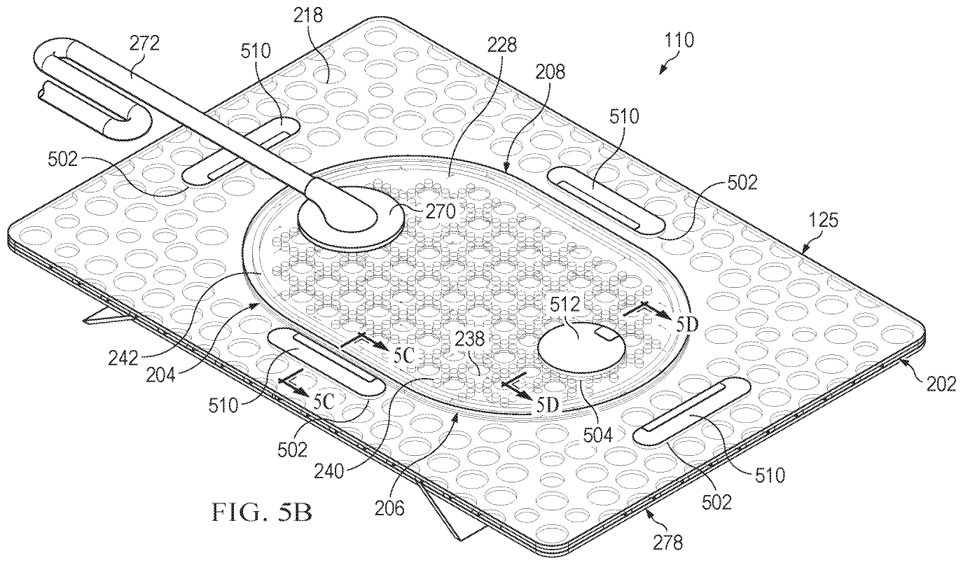

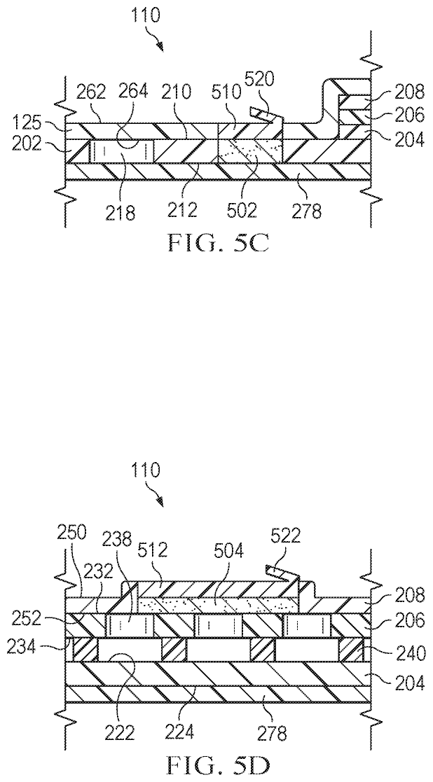

FIG. 5A shows a perspective view of another example of the dressing 110 of FIG. 1, illustrating additional details that may be associated with some embodiments. Similar to FIG. 3, the cover 125, the second film layer 208, the manifold 206, the first film layer 204, and/or the wound contact layer 202 may be substantially clear or optically transparent, allowing for visualization of the layers of the dressing 110 as well as visualization through the windows 238 of the manifold 206.

The dressing 110 may include periwound sensors 502. In some embodiments, the periwound sensors 502 may be disposed on or within the wound contact layer 202. For example, in some embodiments, the periwound sensors 502 may be disposed between the second surface 264 of the cover 125 and the first surface 210 of the wound contact layer 202. In other embodiments, the periwound sensors 502 may be combined or blended into the wound contact layer 202 or located proximate to the second surface 212 of the wound contact layer 202. The periwound sensors 502 may be similar to or equivalent to the sensor 220 described above. For example, the periwound sensors 502 may be configured to sense a parameter at a periwound region of a tissue site such as an oxygen level at the periwound region 414 of the tissue site 402.

In some embodiments, the dressing 110 may further include at least one wound sensor 504. In some embodiments, the wound sensor 504 may be disposed between the second surface 252 of the second film layer 208 and the first surface 232 of the manifold 206. In other embodiments, the wound sensor 504 may be located at a different location within the dressing 110 but may still be located proximate to a wound of a tissue site such as the wound 406 of the tissue site 402. The wound sensor 504 may be similar to or equivalent to the sensor 220 described above. For example, the wound sensor 504 may be configured to sense a parameter at a wound of a tissue site such as an oxygen level at the wound 406 of the tissue site 402.

Referring to FIG. 5B, another embodiment of the dressing 110 of FIG. 5A is shown. The dressing 110 may be similar to the dressing 110 of FIG. 5A but may further include periwound sensor covers 510 and a wound sensor cover 512. The periwound sensor covers 510 and the wound sensor cover 512 may be substantially clear or optically transparent, allowing for visualization of the dressing 110 through the periwound sensor covers 510 and the wound sensor cover 512. The periwound sensor covers 510 may be coupled to or integral with the cover 125 and may be disposed proximate to the periwound sensors 502. The periwound sensor covers 510 may be removably coupled to the cover 125 such that each of the periwound sensor covers 510 can be removed to expose one of the periwound sensors 502 within the dressing 110.

The wound sensor cover 512 may be coupled to or integral with the second film layer 208 and may be disposed proximate to the wound sensor 504 in some embodiments. The wound sensor cover 512 may be removably coupled to the second film layer 208 such that the wound sensor cover can be removed to expose the wound sensor 504 within the dressing 110.

In some embodiments, the periwound sensor covers 510 and the wound sensor cover 512 may be included with the dressing 110 such that the periwound sensors 502 and the wound sensor 504 can be added to or removed from the dressing 110 while the dressing 110 is deployed at a tissue site such as the tissue site 402. The periwound sensor covers 510 and the wound sensor covers 512 may be impermeable to both water vapor and liquids to provide a fluid seal with the cover 125 and the second film layer 208, respectively. The periwound sensor covers 510 and the wound sensor covers 512 may isolate the periwound sensors 502 and the wound sensors 504 from the external or ambient environment so the periwound sensors 502 and the wound sensors 504 may sense parameters at the periwound region 414 and the wound 406 of the tissue site 402.

FIG. 5C is a cross-sectional view of the dressing 110 taken at line 5C-5C. The periwound sensor 502 may be disposed within the wound contact layer 202 and the periwound sensor cover 510 may be coupled to the cover 125. The periwound sensor cover 510 may include a flap 520. The flap 520 may enable a health care provider or another user to easily remove the periwound sensor cover 510 from the dressing 110. When the periwound sensor cover 510 is removed from the dressing 110, the periwound sensor 502 may be accessible through the cover 125.

FIG. 5D is a cross-sectional view of the dressing 110 taken at line 5D-5D. The wound sensor 504 may be disposed proximate to the first surface 232 of the manifold 206. In other embodiments, the wound sensor 504 may be disposed at a different location such as in one of the plurality of windows 238 of the manifold 206. The wound sensor cover 512 may be coupled to the second film layer 208. The wound sensor cover 512 may include a flap 522 similar to the flap 520 of the periwound sensor cover 510.

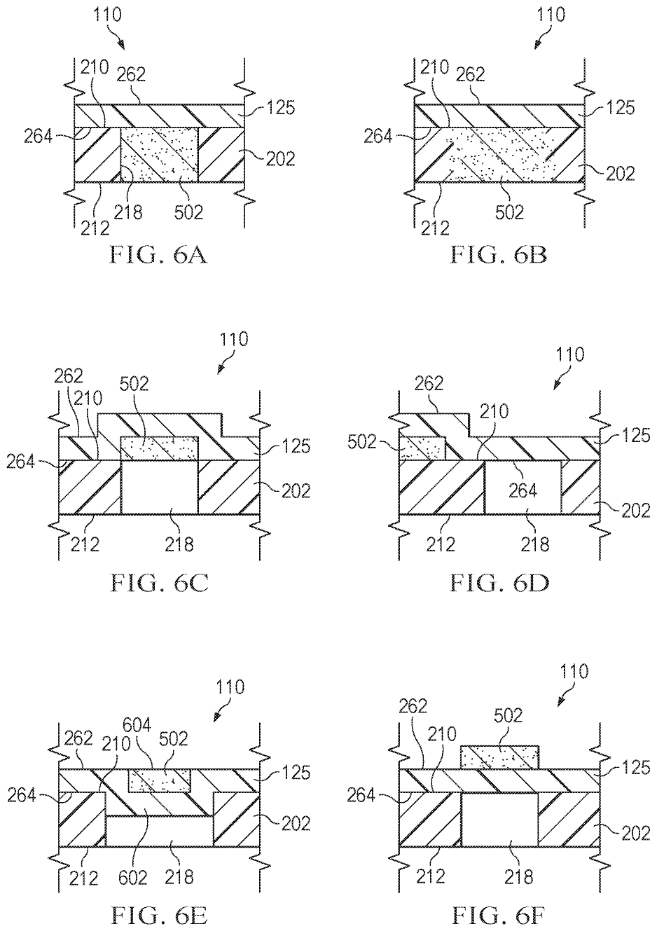

FIGS. 6A-6F are cross sectional views of the dressing 110 showing the periwound sensor 502 disposed at different locations within the dressing 110. FIG. 6A shows the periwound sensor 502 disposed within one of the plurality of apertures 218 of the wound contact layer 202. When the dressing 110 is disposed at a tissue site such as the tissue site 402, the periwound sensor 502 may be in direct contact with the periwound region 414 of the tissue site 402.

FIG. 6B shows the periwound sensor 502 molded or blended into the wound contact layer 202. The periwound sensor 502 may be separated from the plurality of apertures 218 and may be in direct contact with the periwound region 414 of the tissue site 402 when the dressing 110 is disposed at the tissue site 402.

FIG. 6C shows the periwound sensor 502 disposed proximate to the first surface 210 of the wound contact layer 202. The periwound sensor 502 may be disposed proximate to one of the plurality of apertures 218 but may be separated from the periwound region 414 of the tissue site 402 when the dressing 110 is disposed at the tissue site 402.

FIG. 6D shows the periwound sensor 502 disposed between the first surface 210 of the wound contact layer 202 and the second surface 264 of the cover 125. The periwound sensor 502 may be isolated from the plurality of apertures 218 such that the periwound sensor 502 is isolated from the periwound region 414 of the tissue site 402 when the dressing 110 is disposed at the tissue site 402.

FIG. 6E shows the periwound sensor 502 coupled to the first surface 262 of the cover 125 opposite one of the plurality of apertures 218 of the wound contact layer 202. A portion 602 of the cover 125 may be disposed within one of the plurality of apertures 218 such that a first surface 604 of the periwound sensor 502 is flush with the first surface 262 of the cover 125. In some embodiments, the periwound sensor 502 may be isolated from the periwound region 414 of the tissue site 402 to provide a baseline or an atmospheric oxygen level reading when the sensor is acted upon by a light source. In some embodiments, multiple periwound sensors 502 may be used such that a parameter can be measured at multiple locations with the periwound sensors 502.

FIG. 6F shows the periwound sensor 502 coupled to the first surface 262 of the cover 125 opposite one of the plurality of apertures 218 of the wound contact layer 202. Similar to the periwound sensor 502 shown in FIG. 6E, the periwound sensor 502 may be isolated from the periwound region 414 of the tissue site 402 to provide a baseline or an atmospheric oxygen level reading when the sensor is acted upon by a light source. In some embodiments, multiple periwound sensors 502 may be used such that a parameter can be measured at multiple locations with the periwound sensors 502.

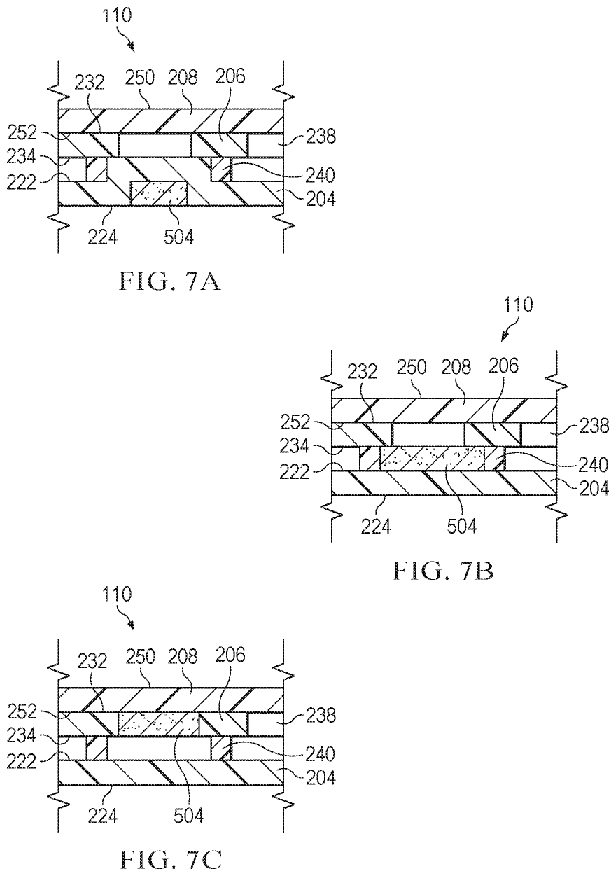

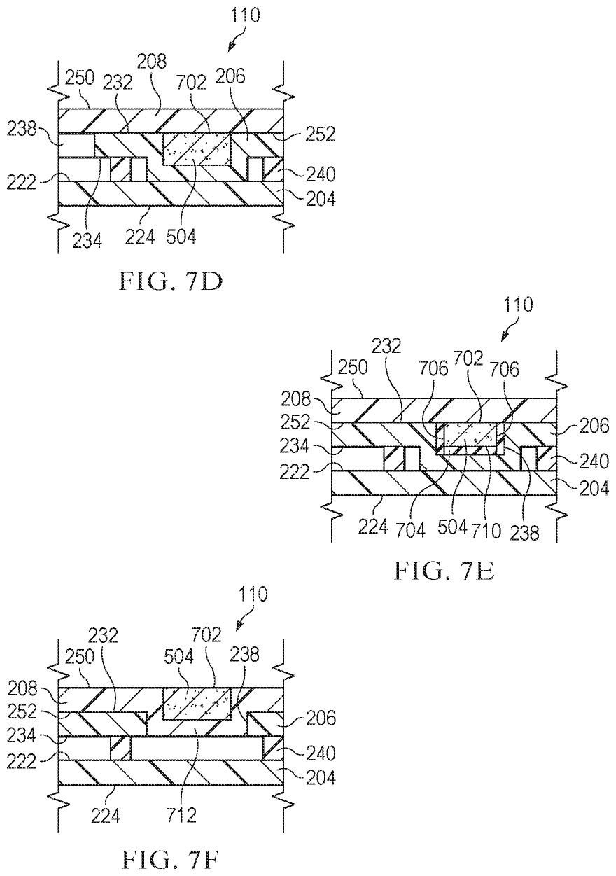

FIGS. 7A-7F are cross sectional views of the dressing 110 showing the wound sensor 504 disposed at different locations within the dressing 110. FIG. 7A shows the wound sensor 504 coupled to the second surface 224 of the first film layer 204. The first film layer 204 proximate to the wound sensor 504 may be pushed into the manifold 206 such that the first surface 222 of the first film layer 204 is coupled to at least one of the plurality of standoffs 240 and the second surface 234 of the manifold 206 such that the wound sensor 504 is flush with the second surface 224 of the first film layer 204. When the dressing 110 is disposed at the tissue site 402, the wound sensor 504 may be in direct contact with the wound 406 of the tissue site 402.

FIG. 7B shows the wound sensor 504 disposed between the manifold 206 and the first film layer 204. The wound sensor 504 may be disposed between the plurality of standoffs 240 and at least a portion of the wound sensor 504 may be proximate to one of the plurality of windows 238 of the manifold 206. When the dressing 110 is disposed at the tissue site 402, the wound sensor 504 may be in fluid communication with the wound 406 of the tissue site 402 through at least the one or more fluid passages 228 of the first film layer 204.

FIG. 7C shows the wound sensor 504 disposed within one of the plurality of windows 238 of the manifold 206. When the dressing 110 is disposed at the tissue site 402, the wound sensor 504 may be in fluid communication with the wound 406 of the tissue site 402 through at least the one or more fluid passages 228 of the first film layer 204.

FIG. 7D shows the wound sensor 504 disposed between the manifold 206 and the second film layer 208. A portion of the manifold 206 may be recessed between the plurality of standoffs 240 of the manifold 206 such that a first surface 702 of the wound sensor 504 is flush with the first surface 232 of the manifold 206. When the dressing 110 is disposed at the tissue site 402, the wound sensor 504 may have fluid contact with the wound 406 of the tissue site 402 through the manifold 206 and the one or more fluid passages 228 of the first film layer 204.

FIG. 7E shows the wound sensor 504 disposed between the manifold 206 and the second film layer 208. A portion of the manifold 206 may be recessed between the plurality of standoffs 240 of the manifold 206 such that the first surface 702 of the wound sensor 504 is flush with the first surface 232 of the manifold 206. The dressing 110 may further include a sealing material 704 disposed around the wound sensor 504 to fluidly isolate the wound sensor 504 from the wound 406. In some embodiments, the wound sensor 504 may have an edge 706 that may couple the first surface 702 of the wound sensor 504 to a second surface 710 of the wound sensor 504. The edge 706 and the second surface 710 of the wound sensor 504 may be coupled to the sealing material. The first surface 702 of the wound sensor 504 may be coupled to the second film layer 208 to fluidly isolate the wound sensor 504 from both the ambient environment and the wound 406.

FIG. 7F shows the wound sensor 504 coupled to the first surface 250 of the second film layer 208 opposite one of the plurality of windows 238 of the manifold 206. A portion 712 of the second film layer 208 may be disposed within one of the plurality of windows 238 of the manifold 206 such that the first surface 702 of the wound sensor 504 is flush with the first surface 250 of the second film layer 208. In some embodiments, the wound sensor 504 may be isolated from the wound 406 of the tissue site 402 to provide a baseline or an atmospheric oxygen level reading when the wound sensor 504 is acted upon by a light source. In some embodiments, multiple wound sensors 504 may be used such that a parameter can be measured at multiple locations with the wound sensors 504.

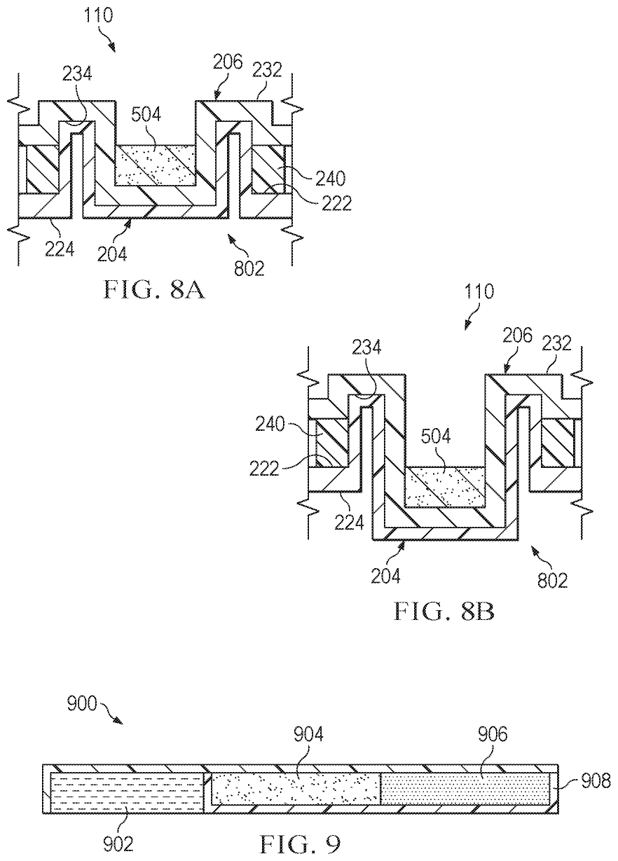

FIGS. 8A and 8B show a portion of an alternate embodiment of the dressing 110 that can be used with deep wounds. In some instances, a wound may have contours or deep regions that require a modified dressing that contains rolling diaphragms 802. As shown in FIGS. 8A and 8B, the rolling diaphragm 802 can include the first film layer 204 and the manifold 206. In other embodiments, the rolling diaphragm 802 may further include the second film layer 208. The wound sensor 504 may be disposed on the first surface 232 of the manifold and may have fluid contact with the wound 406 of the tissue site 402 through the manifold 206 and the one or more fluid passages 228 of the first film layer 204. As shown in FIG. 8B, the rolling diaphragm 802 may extend past the second surface 224 of the first film layer 204 in some embodiments. The rolling diaphragm 802 may allow the dressing 110 to extend into deep wounds so that the wound sensor 504 may be in closer contact with deep regions of the wound. In some embodiments, the dressing 110 may be manufactured such that the rolling diaphragms 802 are spaced out along the dressing 110 so that the wound sensors 504 may be disposed at different depths within the wound that is being treated with the dressing 110.

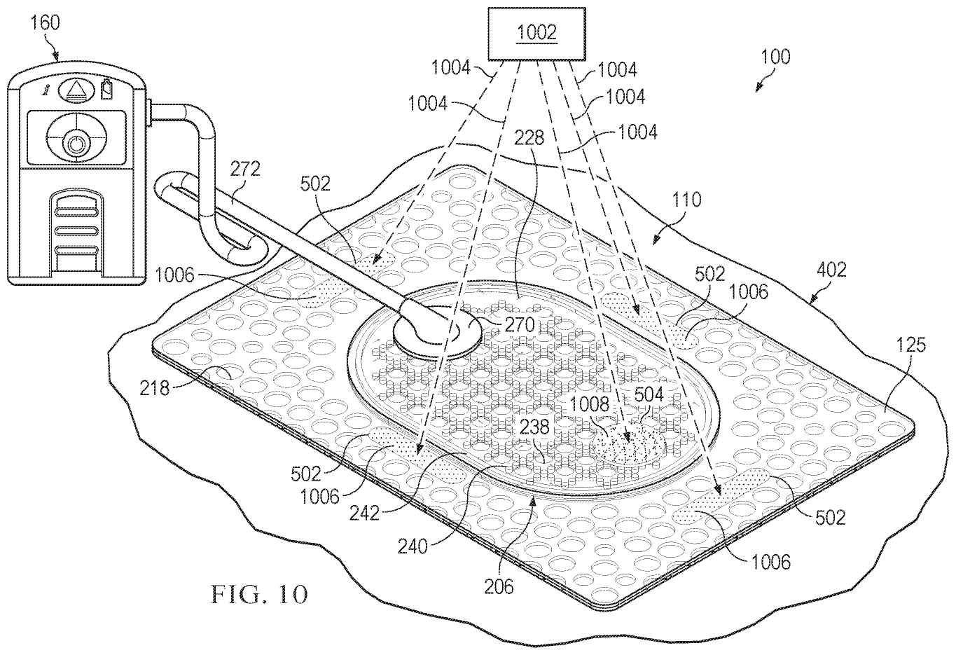

FIG. 9 shows an example of a sensor 900 that may be used with any of the previously described embodiments of the dressing 110. It may be desirable to monitor multiple parameters at a tissue site with one sensor. The sensor 900 may include a fluid sensor 902, an oxygen sensor 904, and a pH sensor 906 in some embodiments. In other embodiments, the sensor 900 may monitor fewer or additional parameters. In some embodiments, there may be a liquid barrier 908 that may be disposed around the oxygen sensor 904 and the pH sensor 906 of the sensor 900. The liquid barrier 908 may keep any liquids from the wound out of direct contact with the oxygen sensor 904 and the pH sensor 906 but allow liquid to come into direct contact with the fluid sensor 902. In some embodiments, the oxygen sensor 904 and the pH sensor 906 may be configured to change color to indicate an oxygen level and a pH level, respectively, when acted upon by a light source. In some embodiments, the intensity of the emitted light may be dependent on the value of the sensed parameter as discussed above with reference to FIG. 2. The fluid sensor 902 may be configured to detect the presence of fluid. The fluid sensor 902 may be partially or fully opaque when dry and may be transparent when exposed to fluid such that when the sensor 220 transitions from opaque to transparent, the sensor 220 has detected the presence of fluid. The sensor 900 may be configured in either a periwound region or a wound contact region of a dressing. In some embodiments, the sensor 900 may be disposed across both the periwound region and the wound contact region such that the fluid sensor 902 may maceration or the presence of fluid in the periwound region which the oxygen sensor 904 and the pH sensor 906 are configured to detect levels of oxygen and pH at the wound of the tissue site.

FIG. 10 shows an example embodiment of the therapy system 100 deployed at the tissue site 402 with a light source 1002 shining on the dressing 110. The light source 1002 may emit light 1004 towards the dressing 110. The light 1004 may penetrate the cover 125 of the dressing 110 to reach the periwound sensors 502 and the wound sensor 504. Once the light 1004 reaches the periwound sensors 502 and the wound sensor 504, the periwound sensors 502 and the wound sensor 504 may change color. The color of the periwound sensors 502 and the wound sensor 504 corresponding to the value of the sensed parameter where they are located. For example, if the sensed parameter is an oxygen level, the periwound sensors 502 and the wound sensor 504 may change color corresponding to the oxygen level that they each sense. The periwound sensors 502 may be a first color 1006 which may indicate a first oxygen level and the wound sensor 504 may be a second color 1008 which may indicate a second oxygen level. The second color 1008 from the wound sensor 504 may be more intense or more vibrant than the first color 1006 which may indicate that the wound sensor 504 is sensing a lower oxygen level than the periwound sensors 502.

In some embodiments, not pictured herein, a UV sensitive CCD camera may be placed above the dressing 110 opposite the tissue site 402 to capture the intensity of the light emitted from the periwound sensors 502 and the wound sensor 504. In some embodiments, not pictured herein, the light source 1002 may include an excitation filter which may concentrate and filter out certain wavelengths of light such that only certain wavelengths reach the periwound sensors 502 and the wound sensor 504 of the dressing 110. Similarly, the UV sensitive CCD camera may include an emission filter in some embodiments to filter and concentrate the light emitted by the periwound sensors 502 and the wound sensor 504.

Also described herein is a method of treating the tissue site 402 with negative pressure. The method may include disposing the dressing 110 on the tissue site 402, applying negative pressure, from the negative-pressure source 105, to the dressing 110, and monitoring the dressing 110 for changes in a sensed parameter at the tissue site 402. The dressing 110 may include the cover 125, the wound contact layer 202, the manifold 206, and at least one sensor such as the sensor 220, the periwound sensor 502 or the wound sensor 504. The cover 125 may include the first surface 262 and the second surface 264. The wound contact layer 202 may include the first surface 210 and the second surface 212. The second surface 212 of the wound contact layer 202 may be configured to contact the tissue site 402. The manifold 206 may include the first surface 232 and the second surface 234. The manifold 206 may be configured to be disposed between the second surface 264 of the cover 125 and the first surface 210 of the wound contact layer 202. The at least one sensor may include a plurality of parameter sensing molecules dissolved in a polymer matrix. The at least one sensor may be configured indicate the sensed parameter when the at least one sensor is acted upon by the light source 1002.

In some example embodiments, monitoring the dressing for changes in the sensed parameter at the tissue site 402 may include detecting a change of color in that at least one sensor when the at least one sensor is acted upon by a light source. In some example embodiments, the sensed parameter may include at least one of a fluid level, an oxygen level, and a pH.

The systems, apparatuses, and methods described herein may provide significant advantages. For example, the dressing 110 may be transparent which may allow a clinician to monitor the tissue site 402 easily while not disturbing the dressing 110. Sensors such as the sensor 220, the periwound sensor 502, and the wound sensor 504 may all aid clinicians in monitoring the tissue site 402 for changes in parameters such as oxygen levels, pH levels, and fluid detection. These sensors may make dressings smarter, as they are able to convey more information about the tissue site 402, without requiring any electrical components to be integrated into the dressing 110.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a cover comprising a first surface and a second surface;
a wound contact layer comprising a first surface, a second surface, and a central aperture, the second surface of the wound contact layer configured to contact a periwound region of the tissue site;
a first film layer comprising a first surface and a second surface, the second surface of the first film layer configured to contact a wound of the tissue site; and
a manifold comprising a first surface and a second surface, the manifold configured to be disposed between the second surface of the cover and the first surface of the first film layer; and
wherein the wound contact layer further comprises at least one sensor comprising a plurality of oxygen sensing molecules dissolved in a polymer matrix and being configured to change color to indicate a sensed oxygen level when the at least one sensor is acted upon by a light source.

2. The dressing of claim 1, wherein the wound contact layer further comprises a peripheral portion surrounding the central aperture, the peripheral portion comprising a plurality of apertures.

3. The dressing of claim 2, wherein the at least one sensor is disposed within one of the plurality of apertures of the wound contact layer.

4. The dressing of claim 2, wherein the at least one sensor is disposed between the first surface of the wound contact layer and the second surface of the cover, the at least one sensor adjacent to at least one of the plurality of apertures of the wound contact layer.

5. The dressing of claim 2, wherein the at least one sensor is disposed between the first surface of the wound contact layer and the second surface of the cover, the at least one sensor offset from the plurality of apertures of the wound contact layer.

6. The dressing of claim 1, wherein the at least one sensor comprises an oxygen sensing film, wherein
the oxygen sensing film comprises a low oxygen permeable polymer or a high oxygen permeable polymer,
the low oxygen permeable polymer comprises one of polyvinyl alcohol, ethylene vinyl alcohol, polyacrylonitrile, and polyvinylidene chloride, and
the high oxygen permeable polymer comprises one of silicone and polyolefin elastomer.

7.-9. (canceled)

10. The dressing of claim 1, wherein the at least one sensor comprises a multi-sensor configured to detect a fluid level, an oxygen level, and a pH level.

11. A dressing for treating a tissue site with negative pressure, the dressing comprising:
a cover comprising a first surface and a second surface;
a wound contact layer comprising a first surface, a second surface, a peripheral portion with a plurality of apertures, and a central aperture, the second surface of the wound contact layer configured to contact a periwound region of the tissue site;
a first film layer comprising a first surface and a second surface, the second surface of the first film layer configured to contact a wound of the tissue site; and
a manifold comprising a first surface and a second surface, the manifold configured to be disposed between the second surface of the cover and the first surface of the first film layer;
wherein the cover further comprises at least one sensor comprising a plurality of oxygen sensing molecules dissolved in a polymer matrix and being configured to change color to indicate a sensed oxygen level when the at least one sensor is acted upon by a light source.

12. The dressing of claim 11, wherein the at least one sensor is disposed within a recess in the first surface of the cover such that the at least one sensor is aligned with one of the plurality of apertures of the wound contact layer.

13. The dressing of claim 12, further comprising a recess lid removably coupled to the first surface of the cover, the recess lid configured to seal the at least one sensor within the recess in the first surface of the cover.

14. The dressing of claim 11, wherein the at least one sensor is disposed on the first surface of the cover such that the sensor is aligned with one of the plurality of apertures of the wound contact layer.

15. The dressing of claim 11, wherein the cover further comprises a central portion with a central aperture and a peripheral portion, the peripheral portion of the cover configured to couple to the peripheral portion of the wound contact layer.

16. The dressing of claim 15, wherein the cover further comprises a second film layer with a first surface and a second surface, the second film layer configured to substantially align with the central aperture of the cover and the second surface of the second film layer configured to couple to the first surface of the manifold.

17. The dressing of claim 16, wherein the at least one sensor is disposed within a recess in the first surface of the second film layer.

18. The dressing of claim 17, further comprising a recess lid removably coupled to the first surface of the second film layer, the recess lid configured to seal the at least one sensor within the recess in the first surface of the second film layer.

19. The dressing of claim 16, wherein the at least one sensor is disposed between the first surface of the manifold and the second surface of the second film layer.

20. The dressing of claim 16, further comprising at least one sealed cavity between the first surface of the manifold and the second surface of the second film layer, the at least one sensor disposed within the at least one sealed cavity.

21.-23. (canceled)

24. A method of treating a tissue site with negative pressure, the method comprising:
disposing a dressing on the tissue site, the dressing comprising:
a cover comprising a first surface and a second surface;
a wound contact layer comprising a first surface and a second surface, the second surface of the wound contact layer configured to contact the tissue site;
a manifold comprising a first surface and a second surface, the manifold configured to be disposed between the second surface of the cover and the first surface of the wound contact layer; and
at least one sensor comprising a plurality of parameter sensing molecules dissolved in a polymer matrix, the at least one sensor configured to indicate a sensed parameter when the at least one sensor is acted upon by a light source;
applying negative pressure, from a negative pressure source, to the dressing; and
monitoring the dressing for changes in the sensed parameter at the tissue site.

25. The method of claim 24, wherein monitoring the dressing for changes in the sensed parameter at the tissue site comprises detecting a change of color in the at least one sensor when the at least one sensor is acted upon by the light source.

26. The method of claim 24, where the sensed parameter comprises at least one of a fluid level, an oxygen level, and a pH.

27. (canceled)

28. (canceled)
